(19) United States
(12) Patent Application Publication
(10) Pub. No.: US 2016/0228380 A1
(43) Pub. Date: Aug. 11, 2016

(54) IMPREGNANATION OF OXYGEN CARRIER COMPOUNDS INTO CARRIER MATERIALS PROVIDING COMPOSITIONS AND METHODS FOR THE TREATMENT OF WOUNDS AND BURNS

(71) Applicant: VIRGINIA COMMONWEALTH UNIVERSITY, Reston, VA (US)

(72) Inventors: Mark A. McHugh, Richmond, VA (US); Rajendar Reddy Mallepally, Richmond, VA (US); Kevin R. Ward, Superior Towship, MI (US); Gary S. Huvard, Richmond, VA (US)

(21) Appl. No.: 15/023,619

(22) PCT Filed: Sep. 23, 2014
(86) PCT No.: PCT/US2014/056913

§ 371 Date: Mar. 21, 2016

Related U.S. Application Data
(60) Provisional application No. 61/881,548, filed on Sep. 24, 2013.
(60) Provisional application No. 61/891,924, filed on Oct. 17, 2013.

(51) Int. Cl. A61K 9/70; A61K 47/38; A61L 15/28; A61L 15/18; A61K 9/06; A61L 15/44; A61K 31/025; A61K 47/02; A61K 45/06; A61K 33/40; A61L 15/42

(52) U.S. Cl. CPC A61K 9/7007; A61K 33/40; A61K 47/38; A61L 15/28; A61L 15/18; A61L 15/425; A61L 15/44; A61K 31/025; A61K 47/02; A61K 45/06; A61K 9/06; A61L 2300/106; A61L 2300/11

(57) ABSTRACT

Oxygen carrier compounds are impregnated into carrier materials that have free volume, empty or void space or high porosity using sub or supercritical fluid assisted processing. Compositions and methods for the treatment of wounds and burns are provided comprising peroxide compounds or perfluorinated compounds impregnated into carrier materials applied directly to the wound or burn.

### FIELD OF THE INVENTION

The invention generally relates to a novel method for the impregnation of oxygen carrier compounds such as hydrogen peroxide into carrier materials that have free volume, empty or void space or high porosity. Aspects of the invention provide compositions and methods for the treatment of wounds and burns. In particular, embodiments of the invention provide CO₂-assisted impregnation of carrier materials for controlled O₂ delivery, for example, hydrogen peroxide loaded cellulose acetate mats or perfluorodecalin loaded silica aerogels applied topically to wounds and burns.

### BACKGROUND OF THE INVENTION

Sub and supercritical CO₂ assisted impregnation has been reported as a viable approach for a variety of substances such as drugs^{1,2}, flavors^{3,4}, dyes⁵ etc. The CO₂ assisted impregnation has a number of advantages, such as processing without organic solvents and uniform distribution of active substance in the matrix. The impregnation process depends on the partitioning of the active substance between the CO₂-rich and polymer-rich phases. Therefore, the relative solubility of the active substance in CO₂ and in the polymer has a significant effect on the amount of active substance loaded into the polymer⁶. Although research has been done in the area of CO₂ assisted impregnation, there are no studies aimed at loading hydrogen peroxide (H₂O₂) using this method. Impregnation of H₂O₂ into carrier materials with high glass transition temperatures (for example, cellulose acetate) by traditional methods (for example, soaking) has shown to be unsuccessful in experiments performed in our laboratories.

H₂O₂ is a widely used chemical oxygen producing compound, because it decomposes into water and oxygen and it carries 47 wt % O₂ per unit mass. H₂O₂ almost instantaneously releases oxygen when contacted with a wound site, due to the catalytic decomposition by an enzyme catalase⁸. Hence, the controlled release of H₂O₂ has the potential to be an effective method for in situ sustained oxygen delivery. Oxygen has been used as a therapeutic agent to speed up healing of acute and chronic wounds^{9,10}. Therefore, an adequate supply of oxygen is important for wound healing.

Perfluorinated compounds or perfluorocarbons (PFC) are chemically and biologically inert substances able to dissolve significant amounts of gases especially O₂, which makes them attractive O₂ carrier materials. A considerable amount of research in the past three decades reports on the use of perfluorocarbon-based emulsions as artificial O₂ carriers. Several studies show the therapeutic benefits of oxygen for healing acute and chronic wounds^{9,10}. Although a common perception is that skin receives O₂ through internal blood circulation, a recent study shows that significant amounts of O₂ penetrate up to ˜700 μm deep into human skin¹¹ from a topically applied source. Davis et al. show how the topical application of a perfluorocarbon emulsion, supersaturated with oxygen, significantly enhances the epithelialization of partial-thickness acute wounds and second-degree burns¹². However, the application of perfluorocarbon emulsions is currently quite limited due to the difficulty in preparing the emulsion and maintaining its stability for extended periods of time. In addition, perfluorocarbons typically exhibit a very short in vivo half-life^{13,14}. Several research groups addressed these deficiencies by synthesizing PFC-filled, core-shell micro- or nano-capsules with a silica or a polymeric shell^{13,15-17}. Although micro- or nano-encapsulation of PFCs is a promising technique, the capsules are susceptible to rupture and loss of PFC.

Topical application of oxygen has been clearly demonstrated to assist in wound healing⁹. However, the potential for such a topical therapy has not been realized due to the cumbersome nature of current gaseous oxygen supply. Attempts have been made to provide bandages that create oxygen, but these have been limited by the small time frame in which oxygen is made¹². U.S. Pat. No. 8,439,860 discloses an oxygen generating wound dressing, however oxygen generation only lasts approximately 20 minutes. It would be highly impractical to change the bandage every 20 minutes for effective treatment. Successful creation of a bandage with long-lasting oxygen delivery would have major implications including the potential to prevent progression of partial thickness burns to full thickness burns.

### SUMMARY OF THE INVENTION

Embodiments of the present invention provide new methods for the impregnation of oxygen carrier compounds into carrier materials that have free volume, empty or void space or high porosity. In exemplary embodiments, the invention provides a method for the impregnation of H₂O₂ or PFC into carrier materials, such as cellulose acetate or silica aerogels using CO₂ assisted processing. Further embodiments of the invention provide new compositions and methods for the treatment of wounds and burns. Exemplary embodiments include cellulose acetate (CA) mats or silica aerogels impregnated with H₂O₂ and PFC respectively that are administered topically for the controlled delivery of O₂ to wounds and burns. In one aspect of the invention, the PFC used is perfluorodecalin (PFD) and the PFD loaded silica aerogel is coated with poly(methyl methacrylate) (PMMA) to dramatically reduce the loss of PFD.

It is an object of the invention to provide a bandage for the treatment of wounds and burns comprising H₂O₂ or PFC impregnated into a carrier material, wherein said carrier material is a synthetic polymer, inorganic or organic material, or inorganic or organic aerogel, said carrier material in contact with a catalyst on one side and an oxygen-impermeable membrane on another side. In exemplary embodiments, an oxygen-permeable membrane or removable oxygen-impermeable membrane may be positioned between the catalyst and carrier material. An additional oxygen-permeable membrane is positioned on the side of the catalyst in contact with the wound or burn. In preferred embodiments, said oxygen-permeable membrane is a medical grade silicone film.

Adequate oxygen delivery to tissues to a degree necessary to maintain a certain degree of aerobic metabolism is necessary for long term survival and normal tissue function after severe injury. The compositions and bandages of the present invention are capable of delivering oxygen to the wound or burn for about 3 days or longer. The materials of the invention are generally non-toxic or easily isolated from tissues, generally inexpensive and readily available, generally biocompatible, and often bioabsorbable.

Other features and advantages of the present invention will be set forth in the description of invention that follows, and in part will be apparent from the description or may be learned by practice of the invention. The invention will be realized and attained by the compositions and methods particularly pointed out in the written description and claims hereof.

### BRIEF DESCRIPTION OF THE DRAWINGS

FIG. 1. Digital image of the bandage made from H₂O₂-loaded CA mats and silicone films (A) and Franz cell experimental set up used for in vitro H₂O₂ release from bandages (B).

FIG. 2. Effect of impregnation temperature on the H₂O₂ loading in CA mats at 1200 psig.

FIG. 3. Effect of H₂O₂—H₂O solution concentration, used for the CO₂ assisted impregnation, on the H₂O₂ loading in CA mats at 25° C. and 1200 psig.

FIG. 4. TGA curves for (A) as received CA mat and 9.5 wt % H₂O₂ loaded CA mat (processed at 25° C. and 1200 psig) and (B) as received H₂O₂—H₂O (50:50 w/w) solution and 9.5 wt % H₂O₂ loaded CA mat on CA mat weight free basis.

FIG. 5. Storage stability of H₂O₂-loaded CA mats at 2-to-8° C. (•) and 20-to-23° C. (□).

FIG. 6. SEM images of CA mats (A) as received and (B) ˜22 wt % H₂O₂-loaded CA mat.

FIG. 7. Images of CA mats after soaking in H₂O₂—H₂O solutions.

FIG. 8. in vitro H₂O₂ release kinetics from the 22 wt % H₂O₂-loaded CA mat (15 mm×15 mm, processed at 25° C., 1200 psi, and 80:20 w/w H₂O₂—H₂O solution used in impregnation) measured in saline at 37° C.

FIG. 9. Schematic diagram of a preferred embodiment of the invention: H₂O₂ impregnation into CA nonwoven mats using a high-pressure CO₂ assisted process.

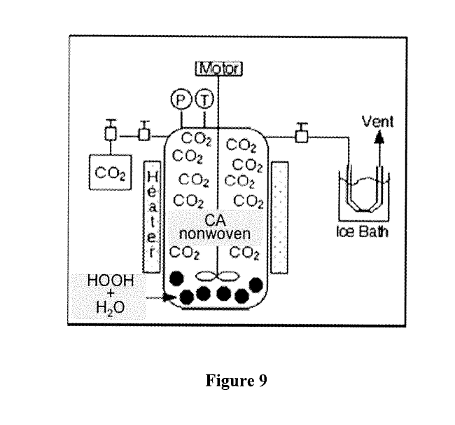

FIG. 10. Schematic diagram of the oil-in-water (O/W) emulsification and solvent evaporation method used to coat aerogel particles with PMMA.

FIG. 11. Vapor-liquid equilibrium of the PFD-CO₂ system at 20° C.¹⁸.

FIG. 12. Effect of contact time on the PFD loading of silica aerogels at 25° C. and 900 psia obtained in this study.

FIG. 13. TGA curves for PFD-loaded, but uncoated aerogel (∘) and unloaded aerogel (⋄); in both the cases the aerogels are heated to 150° C. and held at this temperature for 50 minutes.

FIG. 14. Storage stability of PFD from uncoated aerogels at 2-to-8° C. (∘) and at 20-to-23° C. (□).

FIG. 15. TGA curve for (∘) coated aerogel and (-) sample temperature.

FIG. 16. PFD weight loss comparison from (⋄) coated aerogel, (Δ) uncoated aerogel, and (∘) as received PFD.

FIG. 17. (A) Inverted light microscopy image where PMMA appears dark and PFD-loaded aerogel appears bright and (B) scanning electron microscopy image of coated aerogel.

FIG. 18. Schematic diagram of a preferred embodiment of the invention: a bandage for the treatment of wounds and burns.

### DETAILED DESCRIPTION OF THE INVENTION

Embodiments of the invention relate to the use of a sub or supercritical fluid assisted process to impregnate an active ingredient (such as an oxygen carrier compound) into carrier materials that have high free volume, empty or void space or high porosity. High porosity is a characteristic of a solid material that has many open pores throughout the solid structure such that the volumetric porosity can range from 5 to 99% of the total volume. The pores provide empty or void space that can be filled with another compound or fluid or gas. The term high free volume is recognized by those skilled in the art of polymer science and engineering to characterize the empty space available between polymer chains that make up the solid material that can have a fiber-like, or sheet-like, or sphere-like structure. The free volume between polymer chains is empty and can be filled with another compound or fluid or gas. Those skilled in the art of polymer science and engineering will recognize that the free volume in an amorphous solid polymer material is related to the glass transition temperature of the polymer.

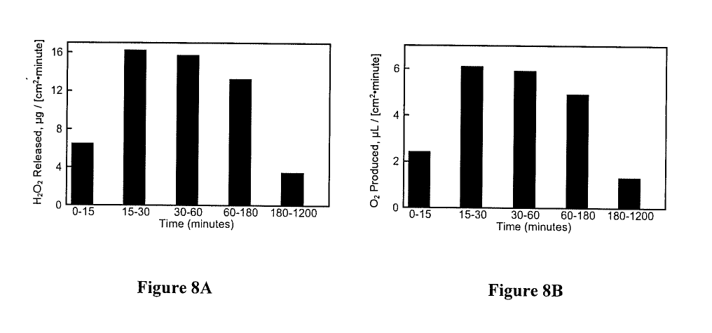

During impregnation as shown in FIG. 8, inside the high pressure apparatus, the supercritical fluid (for example, CO₂) is saturated with the active ingredient (for example, H₂O₂) and passed over the carrier material (for example, cellulose acetate) with mixing at a constant rate. During this process, the active ingredient diffuses into the carrier material and the active ingredient partitions into the available free volume, void volume, free space, and/or pore regions in the carrier material, and the active ingredient is entrapped in the carrier material internal structure, either due to strong specific interactions that attract and hold the active material in the internal structure and/or due to the deposition or condensation or dropping out of carrier (sub- or supercritical) fluid as the system is depressurized.

Aspects of the invention provide for the impregnation of oxygen carrier compounds such as peroxide compounds and PFC. A large amount of oxygen can be dissolved into PFC compounds. The PFC oxygen carrier can be loaded with oxygen either before or after being impregnated into the carrier material by contacting PFC with a source of oxygen, for example atmospheric air.

Other active ingredients which are soluble in sub or supercritical fluids in small fractions can also be impregnated using this technique. The active ingredients can be hydrophilic such as H₂O₂, hydrophobic/lipophilic such as fats, oils, and hydrophobic/lipophobic such as perfluorocarbons. Suitable peroxide compounds of the claimed invention include hydrogen peroxide, sodium peroxide, calcium peroxide, magnesium peroxide, urea hydrogen peroxide, sodium percarbonate, and poly(vinyl pyrrolidone)/hydrogen peroxide complex.

Aspects of the invention relate to one type of carrier material that has a high glass transition temperature characteristic of a material with a frozen, non-equilibrium structure containing empty space (free volume or void volume) between the primary compounds or polymeric chains comprising the carrier material. A high glass transition temperature is defined herein to mean a glass transition temperature of about 100° C. to about 270° C. or higher, preferably a temperature above about 150° C. For example, cellulose acetate polymers are known to have a glass transition temperature near 190° C. which indicates that the cellulose acetate polymer exhibits solid like characteristics with a somewhat rigid structure, but this structure is not crystalline rather the structure is amorphous with substantial unoccupied free volume between polymer chains. Another type of carrier material is a highly porous solid material created from a sol-gel process and further processed using a sub or supercritical fluid solvent to create a highly porous aerogel. Those skilled in the art of supercritical processing will recognize the well-known technique of processing gels and other materials with sub and supercritical fluids to create highly porous solid carrier materials.

Examples of the high pressure apparatus or instruments used in the invention are known in the art and can be purchased commercially. Examples of sub or supercritical fluids used to dissolve the preferred active ingredient and transport that oxygen carrier compound into the carrier material include but are not limited to carbon dioxide (CO₂), ethane, propane, xenon, krypton, and fluorinated compounds that have a critical temperature ranging from −50° C. to 130° C.

In some aspects of the invention, carrier materials are synthetic polymers, inorganic or organic material, or inorganic or organic aerogels. Examples of suitable carrier materials of the invention include but are not limited to:

Synthetic polymers containing a significant portion of polar repeat groups such as polyvinylpyrrolidone) (PVP), poly(ethylene glycol) (PEG), poly(2-ethyl-2-oxazoline), copolymers of ethylene and vinyl acetate, ethylene and methyl acrylate, lactic and glycolic acid, and ter-block copolymers of ethylene oxide blocks followed by propylene oxide blocks followed by ethylene oxide blocks (PEO-PPO-PEO).

Inorganic carriers such as Aerosil® 300, Aerosil® R972, Aeroperl 300 Pharma© which are granulated fumed silicas and are commercially available from Evonik Industries, USA. These non-porous, high surface area silica materials have been used to improve the dissolution characteristics of poorly water soluble drugs. Silica nano particles with defined pore size are prepared using the Stöber process.

Inorganic aerogels such as silica aerogels are prepared using the sol gel process. The high surface area and open pore structure of silica aerogels make them as potential carriers for variety of active ingredients. Table 1 lists the typical textural properties of silica carriers used in this invention.

TABLE 1
Textural characteristics of silica carriers determined
from N₂ adsorption/desorption analysis.

| | Specific surface area | Pore size | Pore volume |
|---|---|---|---|
| Carrier | (m²/g) | (nm) | (cc/g) |
| Aerosil 300 | 280 | none | none |
| Silica Aerogel | 600 | 2-20 | 1.3 |
| SiO₂ nano | 650 | 3-4 | 0.9 |
| particles | | | |

In exemplary embodiments, the active ingredient is H₂O₂ or PFC, the carrier material is silica aerogels or cellulose acetate mats, and CO₂ is the sub or supercritical fluid used to dissolve and transport the preferred oxygen carrier compound into the carrier material where the oxygen carrier compound is then deposited into the carrier material. The preferred PFC is perfluorodecalin (PFD). Methods of the claimed invention produce up to about 45 wt %, e.g. about 5, 10, 15, 20, 25, 30, 35, 40, 45 wt % of active ingredient impregnated into carrier materials. The amount or wt % of active ingredient impregnated into carrier materials can be adjusted through general optimization of the operating conditions of the high pressure apparatus, for example, adjusting the temperature or pressure which adjusts the solvent strength of the sub or supercritical fluid or by adjusting the processing time used to contact the carrier material with the sub or supercritical fluid loaded with the preferred oxygen carrier compound. The high pressure apparatus is operated at a pressure high enough to increase the solvent power of the sub or supercritical fluid so that the preferred oxygen carrier material dissolves or disperses to some extent in the sub or supercritical fluid. Typical operating pressures start at about 500 psi and are up to about 5,000 psi or higher, preferably within a range of about 800 to 1400 psi. At very high pressures, the sub or supercritical fluid will become an effective solvent for dissolving the preferred oxygen carrier compound, however the sub or supercritical fluid solvent power is too high to allow the preferred oxygen carrier compound to partition to any great amount into the carrier material efficiently. The length of time that the apparatus remains pressurized can also be varied, from 1 minute to 24 hours or 2 days and beyond, or more preferably between 30 to 120 minutes. The apparatus is maintained at a temperature of about −20° C. to about 100° C., preferably within a range of about 25° C. to about 40° C.

In preferred embodiments, the impregnated carrier material is coated with polymethyl methacrylate (PMMA), poly(vinylacetate), PLGA copolymer, PLA homopolymer, or other suitable barriers to reduce the loss of the active ingredient impregnated into said carrier material. Coating of the carrier material may be accomplished through several methods, including but not limited to, solvent evaporation techniques, spray drying, coating using a Wurster coater, dip coating, oil-water emulsion techniques, etc.

Methods of the present invention have a variety of applications in diverse industries which include but are not limited to the food, pharmaceutical, and consumer industries. For example, techniques of the invention may be used to develop pharmaceutical drug carriers capable of releasing a drug in a specific location at a specific rate.

Embodiments of the invention pertain to compositions and methods for delivering oxygen to an area or location of interest, e.g. to wounds or burns to promote recovery of the wounds and burns. Aspects of the invention relate to a composition for the storage and controlled release of oxygen (O₂), e.g. for the treatment of wounds and burns, comprising peroxide compounds or PFC impregnated into a carrier material, wherein said carrier material is, for example, a synthetic polymer, inorganic or organic material, or inorganic or organic aerogel, for example silica aerogels, polysaccharide aerogels, and cellulose acetate mats or fibers.

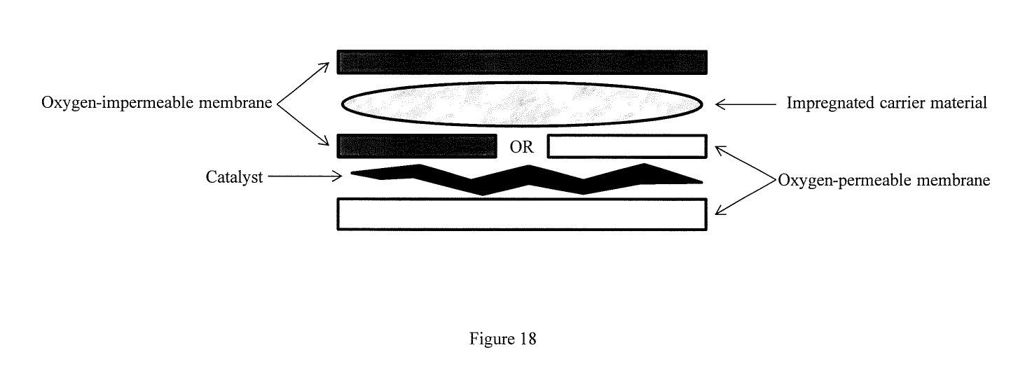

As shown in FIG. 18, aspects of the invention also relate to a bandage for the treatment of wounds and burns comprising a peroxide compound or PFC impregnated into a carrier material, wherein said carrier material is a synthetic polymer, inorganic or organic material, or inorganic or organic aerogel, said carrier material in contact i) on one side with an oxygen-impermeable membrane (e.g. a first oxygen-impermeable membrane) and ii) on another (second) side with an oxygen-permeable membrane, or alternatively, with a second oxygen-impermeable membrane. As described elsewhere herein, the second oxygen-impermeable membrane is generally removable, e.g. prior to or during use of the bandage.

Suitable peroxide compounds of the claimed invention include hydrogen peroxide, sodium peroxide, calcium peroxide, magnesium peroxide, urea hydrogen peroxide, sodium percarbonate, and poly(vinyl pyrrolidone)/hydrogen peroxide complex. In exemplary embodiments, said peroxide compound is H₂O₂. In some embodiments, a catalyst is positioned between said carrier material and oxygen-permeable membrane. Bandages of the claimed invention may also comprise a second oxygen-permeable membrane or a removable oxygen-impermeable membrane between said carrier material and said catalyst.

For example, during use, an oxygen-impermeable membrane between the carrier material and catalyst is removed by sliding the membrane out, thus allowing the impregnated carrier material to come in contact with the catalyst. Additionally, the bandage may be prepared in two parts and combined when ready for use. One part may contain the impregnated carrier material surrounded by oxygen-impermeable membranes and a second part may contain the catalyst and an oxygen-permeable membrane. Before use, an oxygen-impermeable membrane is peeled off of the carrier material and the two parts are combined so that the carrier material contacts the catalyst. In some embodiments, the oxygen-impermeable membrane may be biodegradable or degrade upon contact with a catalyst or other compound. The combination and position of permeable or impermeable membranes used in the bandage are readily recognized by those skilled in the art of creating mass transfer devices for delivering a compound in a preferred direction for a specific time duration.

Further embodiments of the invention relate to a bandage for the treatment of wounds and burns comprising microcapsules, creams, or gels loaded with a peroxide compound or PFC in contact on one side with an oxygen-impermeable membrane and on another side an oxygen-permeable membrane. In exemplary embodiments, said peroxide compound is H₂O₂. In some embodiments, a catalyst is positioned between said carrier material and oxygen-permeable membrane. The bandage may further comprise a second oxygen-permeable membrane or a removable oxygen-impermeable membrane between said microcapsules, creams, or gels and said catalyst. The bandage may further comprise a carrier material positioned on either side of said microcapsules, creams, or gels loaded with a peroxide compound or PFC.

Examples of oxygen-permeable membranes include, but are not limited to, medical grade films made of silicone, polyurethane, polyethylene, polyester, etc. In preferred embodiments, medical grade silicone film is used.

Catalysts of the claimed invention aid in the breakdown of oxygen carrier compounds to result in the release of oxygen from said compound. For example, after contact with a suitable catalyst, peroxide compounds such as H₂O₂ will degrade to release oxygen and water. Suitable catalysts include, but are not limited to, manganese dioxide, silver oxide, iron oxide, copper oxide, platinum oxide, etc., non-metallic catalysts such as potassium iodide and biological catalysts such as catalase. In preferred embodiments, the catalyst is manganese dioxide. In some embodiments, a catalyst is not required for the release of oxygen from oxygen carrier compounds, for example PFC.

The compositions and bandages of the claimed invention may also comprise antimicrobials, vasoactive peptides, anesthetic agents, hemostatic agents, anti-inflammatants, pain-reducing medication, additives to enhance blood clot strength, and/or growth factors can be impregnated into said carrier material or coated onto the said carrier material to act in concert with the oxygen for wound or burn treatment.

It is an object of the invention to provide a method for treating wounds and burns comprising the step of administering to the wound or burn, for example by topical application, a composition comprising an oxygen carrier (such as a peroxide compound or PFC) impregnated into a carrier material, wherein said carrier material is a synthetic polymer, inorganic or organic material, or inorganic or organic aerogel, wherein said peroxide compound or PFC delivers oxygen to the wound or burn. In exemplary embodiments, said peroxide compound is H₂O₂.

Another embodiment of the invention provides a method for treating wounds and burns comprising the step of administering to the wound or burn a bandage comprising a peroxide compound or PFC impregnated into a carrier material, wherein said carrier material is a synthetic polymer, inorganic or organic material, or inorganic or organic aerogel, said carrier material is in contact on one side with an oxygen-impermeable membrane and on another side an oxygen-permeable membrane. In exemplary embodiments, said peroxide compound is H₂O₂. In some embodiments, a catalyst is positioned between said carrier material and oxygen-permeable membrane. Bandages of the claimed invention may also comprise a second oxygen-permeable membrane or a removable oxygen-impermeable membrane between said carrier material and said catalyst. The sustained release of oxygen in the methods of the invention last for about 3 days or longer. In some embodiments, two bandages may be applied sequentially to the wound or burn: a first bandage comprising a catalyst and a second bandage comprising a peroxide compound impregnated into a carrier material. The bandages may include oxygen permeable and impermeable membranes as described above.

The carrier materials described herein are impregnated with oxygen generating substances, e.g. peroxide containing compounds which release O₂ upon breakdown, or substances which are able to dissolve significant amounts of a gas of interest (e.g. O₂) such as PFCs. While the impregnated carrier materials may be used as bandages to deliver O₂ as described elsewhere herein, their use is not limited to this aspect. The materials may be used to store and deliver breathable O₂ to any desired location of interest. Locations of interest include any environment or space into which it is advantageous to provide gaseous O₂, including but not limited to: inside enclosed areas where access to oxygen is limited (e.g. space suits, space stations, diving gear, underwater vehicles, etc.); or where it is desired to augment oxygen levels (green houses, inside oxygen tents); etc. Further, the materials may be used in situations where it is desirable to have a portable supply of stored, readily releasable oxygen, but where other sources of oxygen (e.g. oxygen lines or tanks) are not available, e.g. in emergency circumstances where it would be beneficial to provide oxygen to a subject such as an accident or heart attack or wounded victim on the battlefield, at accident sites, in remote areas, etc. In such cases, the oxygen releasing materials may be used, for example, as a temporary source of O₂ until victims are transported to a treatment center.

The invention will be further illustrated by the following examples. However, these examples and figures should not be interpreted in any way as limiting the scope of the present invention.

### EXAMPLES

### Example 1

### Hydrogen Peroxide Loaded Cellulose Acetate Mats as Controlled Topical O₂ Delivery Devices

This study demonstrates the effect of impregnation process parameters on the H₂O₂ loading and also presents in vitro performance of the H₂O₂-loaded cellulose acetate (CA) mats such as H₂O₂ release, O₂ production rate, and shelf-life of the product. In this study, sub and supercritical CO₂ is used as an effective solvent to dissolve hydrogen peroxide (H₂O₂), to transport the H₂O₂ into the CA mat, and to impregnate CA non-woven mats with hydrogen peroxide (H₂O₂). As much as 30 wt % H₂O₂ can be impregnated into the CA mat at operating conditions of 25-to-40° C. and 1,200-to-1,400 psi. Thermal Gravimetric analysis (TGA) shows that H₂O₂-loaded CA mats lose more than 60% of available H₂O₂ when quickly heated to 37° C. and held at this temperature for 60 minutes. However, in comparison, pure H₂O₂—H₂O (50:50 w/w) solution evaporates within 20 minutes at this same temperature. Shelf-life studies show that H₂O₂-loaded CA mats lose ˜50% and ˜100% of available H₂O₂ in 30 days, when stored at 2-to-8° C. and 20-to-23° C., respectively. H₂O₂ slowly diffuses and subsequently evaporates from the CA mat at a rate determined by the diffusion of H₂O₂ through CA mat. A thin polymer coating can minimize the loss of H₂O₂ by hindering the diffusion of H₂O₂ from the CA mat, thus increasing the shelf-life of the product. These mats are used as bandages for the continuous delivery of O₂ for advanced wound care. In vitro studies show that the developed bandages produce O₂ in a controlled rate for 24 hours. The findings of this study provide insight into the application of supercritical fluid technology as a viable approach to load H₂O₂ into different carriers.

### Materials

H₂O₂—H₂O solutions, 30:70 and 50:50 wt/wt, are purchased from Sigma Aldrich, USA. Concentrated H₂O₂—H₂O solution, 80:20 wt/wt, is obtained by evaporating H₂O from 50:50 wt/wt H₂O₂—H₂O solution in vacuum at 25° C. Cellulose acetate (CA) non-woven mats are donated by Celanese, USA.

### Methods

CO₂-Assisted H₂O₂ Impregnation

A high-pressure apparatus (Parr instruments, model 5500 with magnetic stirrer drive), with different size vessels, is used for CO₂ assisted impregnation. H₂O₂—H₂O solution is placed at the bottom of the vessel. CA mats, dried at 100° C. for two hours, are loaded into a tea bag that is tied to the impeller shaft to keep the CA mat from contacting and soaking in the H₂O₂—H₂O solution. The weight ratio of H₂O₂—H₂O solution to CA mats is ten. Then the vessel is pressurized with CO₂ to 1200±50 psig and temperature is maintained at 25° C. using an externally mounted heating band. The system is maintained at these conditions for 60 minutes with mixing of the H₂O₂—H₂O solution as well as the CO₂-rich gas phase above the solution. During this constant temperature-pressure mixing time the CO₂-rich gas phase dissolves H₂O₂—H₂O and this gas phase penetrates into the CA fibers that make up the mat. The H₂O₂ preferentially interacts with the functional groups on the CA molecule and the H₂O₂ partitions into the free volume of CA fiber. After 60 minutes the CO₂-rich gas phase is slowly vented over a period of 15 minutes from the high-pressure vessel to slowly depressurize the vessel. The H₂O₂—H₂O loaded CA mat is recovered and it is noted that the mat is essentially dry since the H₂O₂ is impregnated in the inner structure of the CA polymeric fibers and the CO₂ releases as a gas at near ambient pressures. The H₂O₂ loading in the mat is determined by recording the CA mat weight before and after impregnation and the loading is verified using thermal gravimetric analysis in a manner recognized by those skilled in the art of this analytical technique.

Characterization of H₂O₂-Loaded CA Mats

H₂O₂ is extracted from H₂O₂-loaded CA mats by suspending in 1:10 v/v sulfuric acid-distilled water solution for 15 minutes with moderate stirring. H₂O₂ concentration of the solution is determined using permanganate titration (HANNA Instruments, HI 902C, USA). The standard deviation of H₂O₂ concentration is ˜2% as determined from three repeated titrations of representative H₂O₂-loaded CA mats.

The H₂O₂—H₂O loading in the CA mats is determined using thermal gravimetric analysis (TGA) (Perkin-Elmer USA, Model Pyris 1 TGA). The furnace is continuously flushed with nitrogen gas at a flow of 3 L/hour. The H₂O₂-loaded CA mats are heated at a rate of 100° C./minute to 37° C. and held at this temperature for 60 minutes. The mats are then quickly heated to 100° C. at a heating rate of 100° C./minute and held at this temperature for 30 minutes.

The morphology of the CA polymeric fibers that compose the mats is determined using scanning electron microscopy (SEM) (HITACHI SU-70). CA fibers, peeled from representative mats, are spread on a graphite paste and then a 5 nm platinum coating is applied via spun coat (Denton Vacuum, LLC, USA, Model: Desk V TSC) onto the sample before capturing the images.

In Vitro H₂O₂ Release Studies

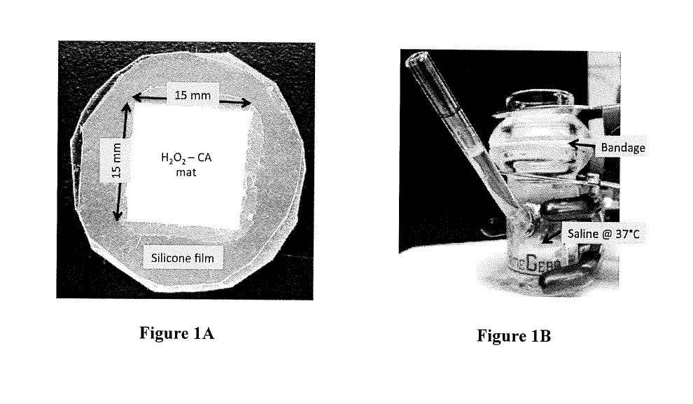

The bandages are made by placing H₂O₂-loaded CA mat between medical grade silicone films and FIG. 1A shows the digital image of the bandage. A Franz cell is used to determine the in vitro H₂O₂ release kinetics from H₂O₂-loaded CA mats. The cell is filled with 15 mL of saline, 0.9 wt % NaCl in distilled water, and maintained at 37° C. The bandage is mounted as depicted in FIG. 1B that mimics the in vivo skin attachment. The saline is stirred at 100 rpm and aliquots drawn at a pre-defined time points are assayed for H₂O₂ release using permanganate titration.

### Results and Discussion

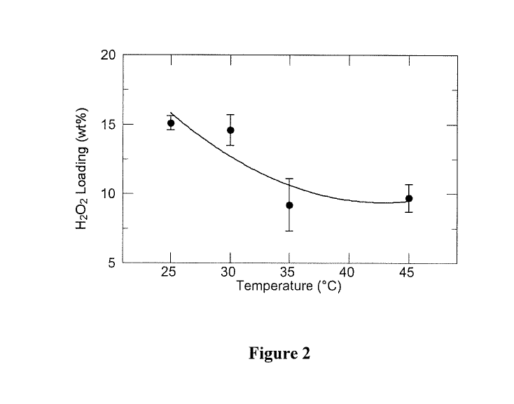

CO₂ assisted impregnation experiments are performed with H₂O₂—H₂O (50:50 w/w) solution at a fixed pressure of 1200 psig and temperatures from 25 to 45° C. The amount of H₂O₂—H₂O used in all four experiments reported here is approximately an order of magnitude higher than the saturation solubility of H₂O₂—H₂O in CO₂. The calculations are based on the pure water solubility in high pressure CO₂, since the solubility of H₂O₂—H₂O in high pressure CO₂ is not available in the literature. FIG. 2 shows four independent experiments that illustrate the effect of temperature on the H₂O₂ loading in CA mats. As the impregnation temperature used in the process is increased, the H₂O₂ loading in the CA mat decreases. The higher H₂O₂ loading at low temperatures is likely due to the higher partitioning of H₂O₂ from CO₂-rich phase to the CA mat, which is likely due to strong interactions between the molecular groups on the CA polymers and the H₂O₂ that are favored at low temperatures. However, the H₂O₂ loading in the CA mat is approximately the same at 25-to-30° C. and 35-to-45° C. The error bars of the data points are the standard deviation of H₂O₂ loading in three independent samples performed in the same experiment to load the CA mats. The results suggest that lower temperatures are favorable for the H₂O₂ loading into CA mats using the CO₂ process.

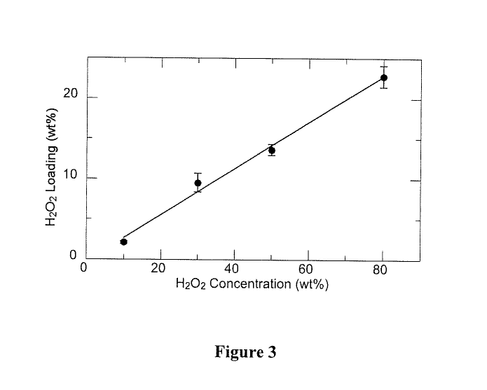

To manipulate the H₂O₂ loading into the CA mats, the impregnation experiments are performed at 25° C. and 1200 psig using four different concentrations of H₂O₂—H₂O solutions. Note that the amount of the H₂O₂—H₂O solution added to the high-pressure vessel at the start of all four experiments is the same and of sufficient amount to maintain the CO₂-rich phase saturated during the impregnation process. As shown in the results for four independent experiments depicted in FIG. 3, the H₂O₂ loading into CA mats increases linearly as the H₂O₂ concentration increases in the starting solution loaded into high-pressure vessel at the start of the impregnation process. The overall conclusion is that H₂O₂ loading into CA mats can be tailored by manipulating the CO₂ assisted impregnation process parameters.

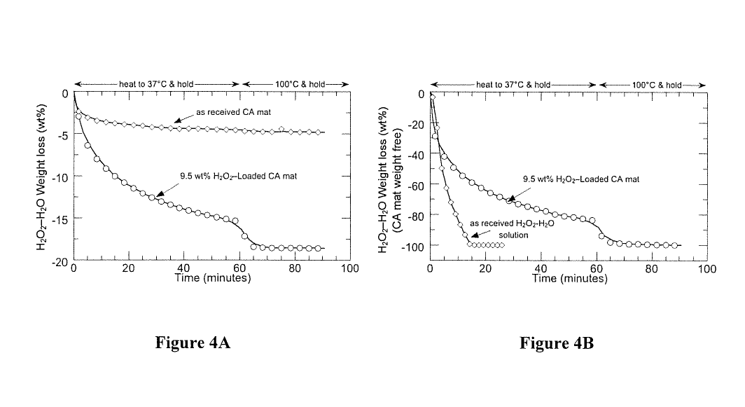

TGA analysis is used to determine the H₂O₂—H₂O loading in the CA mats. FIG. 4 shows the typical H₂O₂—H₂O weight loss from the 9.5 wt % H₂O₂-loaded CA mats. FIG. 4A compares the weight loss from the H₂O₂-loaded CA mats with that of the as-received CA mats. The results show that CA mats are loaded with ˜19 wt % of H₂O₂—H₂O whereas as received CA mat has ˜5 wt % absorbed moisture or other volatiles. FIG. 4B compares the weight loss from two independent TGA analyses where one TGA analysis was performed with the H₂O₂-loaded CA mats containing ˜19 wt % of H₂O₂—H₂O and the other TGA analysis was performed with as received H₂O₂—H₂O (50:50 w/w) liquid solution. The results show that H₂O₂-loaded CA mats lose more than 60% of available H₂O₂ when quickly heated to 37° C. and held at this temperature for 60 minutes. However, in comparison, 100% of H₂O₂—H₂O (50:50 w/w) solution evaporates within 15 minutes at this same temperature. These results indicate that the H₂O₂—H₂O solution is impregnated into the CA fibers of the mat and not just on the outside of the fibers. Had the H₂O₂—H₂O been on the outside of the CA fibers the loss profiles in FIG. 4B would be almost indistinguishable from one another.

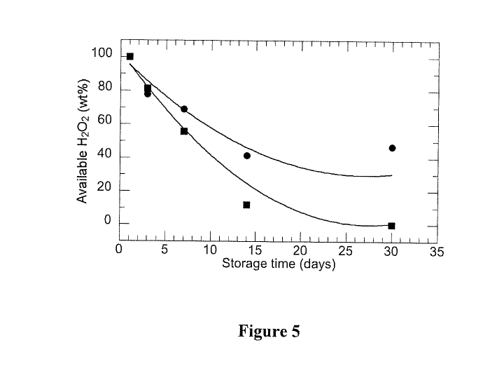

FIG. 5 shows the shelf-life of H₂O₂-loaded CA mats determined at 2-to-8° C. and 20-to-23° C. Approximately 50 wt % of the loaded H₂O₂ is available in the CA mats when stored at 2-to-8° C. for 30 days, whereas the mats stored at 20-to-23° C. lose 100 wt % of loaded H₂O₂ in the same time period. H₂O₂ slowly diffuses from the internal space or free volume of the CA fibers and subsequently evaporates from the surface of the CA mats. The rate of loss of available H₂O₂ from the mats is fixed by the diffusion of H₂O₂ through the CA fibers that make up the mat. It is expected that a thin polymer coating will minimize the loss of H₂O₂, and, hence, increase the shelf-life of the product. Typical over the counter 3 wt % H₂O₂—H₂O solution shelf-life is significantly longer compared to the H₂O₂-loaded CA mats, which is due to the presence of H₂O₂ stabilizers in the solution and due to the cap on the bottle that minimizes vapor losses of H₂O₂. The thin polymer film on the outside of the H₂O₂-loaded CA mat suppresses the loss of H₂O₂ from the mat in a similar manner as the cap on the bottle of 3 wt % H₂O₂—H₂O solution.

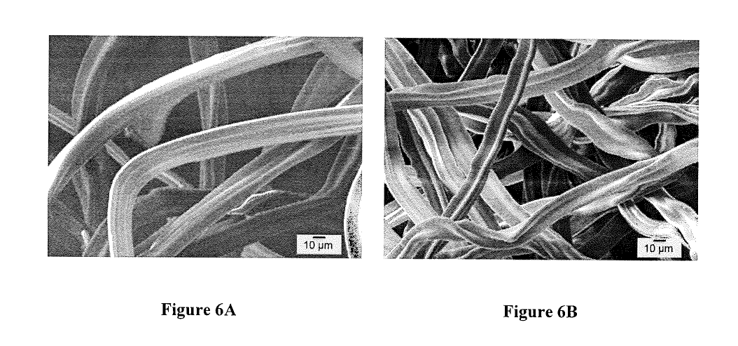

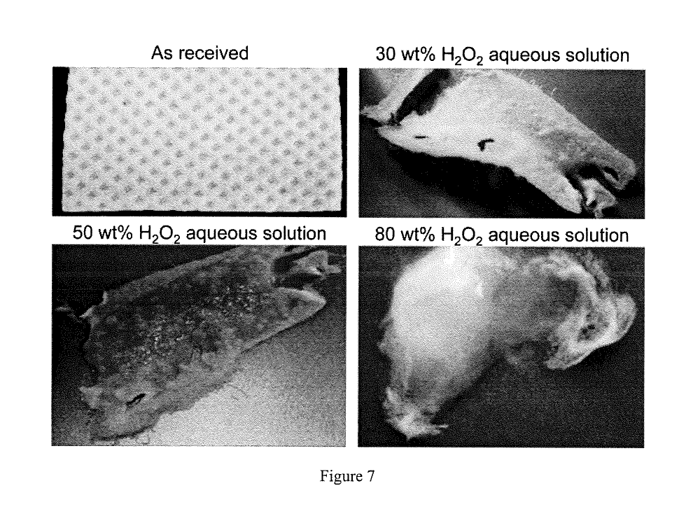

FIG. 6 shows SEM images of CA mats before and after H₂O₂ loading. The morphology of the H₂O₂-loaded CA mat (FIG. 6B) looks similar to that of the virgin CA mat (FIG. 6A), which indicates that the CO₂ assisted impregnation process has no effect on the morphology of the mats. FIG. 7 shows that CA mats lose their structural integrity if soaked directly in H₂O₂—H₂O solutions at H₂O₂ concentrations greater than 30 wt %. Hence direct soaking of CA mats in the H₂O₂—H₂O solutions is not a viable option for loading H₂O₂ into the mats. Therefore, H₂O₂ impregnation using a CO₂ assisted process offers a significant potential for creating novel materials for advanced wound care applications.

FIG. 8 compares the H₂O₂ release rate and corresponding calculated O₂ production rate from the H₂O₂-loaded CA mats. Both H₂O₂ release and corresponding O₂ production are normalized for a 1.0 cm² size CA mat per minute within the time range shown on the abscissa. The O₂ production in FIG. 8B is calculated assuming one-half mole of O₂ is created from the decomposition of a mole of H₂O₂ and using the ideal gas equation at 37° C. and 1 atm. The total O₂ production from the H₂O₂-loaded CA mats is an order of magnitude higher than the typical skin O₂ consumption, which is ˜0.25 μL per cm² of skin. However, the O₂ production can be tailored by manipulating the amount of H₂O₂ loaded into the CA mats. The H₂O₂-loaded CA mats developed in this study can be used for topical oxygenation, needed for wound healing, given the available total O₂ production and the sustained O₂ release for long periods of time.

### Conclusion

In this study CA mats are successfully impregnated with H₂O₂ using sub and supercritical CO₂ at mild operating temperatures and pressures. The H₂O₂ loading in the CA mats can be tailored between 2-to-25 wt % by manipulating the impregnation process parameters. Maximum H₂O₂ loading into CA mats is achieved at temperatures of 25-to-30° C., which is likely due to the higher partitioning of H₂O₂ from CO₂-rich phase to CA mats. Shelf-life studies show that storing the H₂O₂-loaded CA mats at 2-to-8° C. retains more than 50 wt % of the loaded H₂O₂ and thus storing in the refrigerator is preferred over room temperature. The CO₂ assisted impregnation process had no effect on the morphology of the CA fibers that make up the mats. in vitro release studies reveal that sustained H₂O₂ release and, thus, O₂ production is achieved for about 24 hours, using the bandages created from H₂O₂-loaded CA mats. We found that these mats have potential applications for advanced wound care.

### Example 2

### CO₂-Assisted Perfluorodecalin Impregnation into Silica Aerogels

In the present study, silica aerogels are impregnated with perfluorodecalin (PFD) using CO₂-assisted processing to create effective topical oxygen delivery devices. These studies demonstrate the effect of process parameters on the level of PFD impregnation and the minimization of evaporative losses of PFD. Although as much as 45 wt % PFD can be impregnated at mild operating conditions of 25° C. and 900 psia, thermogravimetric analysis (TGA) shows that PFD-loaded silica aerogels lose more than 80% of available PFD when maintained at 37° C. for 60 minutes. Shelf-life studies also show that PFD-loaded silica aerogels lose ˜10% and ˜30% of available PFD in 30 days, when stored in closed bottles at 2-to-8° C. and 20-to-23° C., respectively. To dramatically reduce the loss of PFD, the loaded aerogels are coated with PMMA using a solvent evaporation method. PMMA-coated, PFD-loaded silica aerogels retain 100% of the PFD for up to 30 days when stored at ambient conditions in a closed bottle. The coated aerogels lose less than 20% of the available PFD when maintained at 37° C. for 60 minutes and an additional 10% when heated further to 100° C. and held at this temperature for 30 minutes. The remaining 70% PFD is lost rapidly when the temperature is increased to 150° C., which is slightly above the boiling point of PFD and well above the glass transition temperature of PMMA. These materials offer a method for sustained topical oxygen delivery.

### Materials

Perfluorodecalin, is purchased from SynQuest labs, USA. Tetraethylorthosilicate (TEOS), Methanol, dichloromethane are purchased from Fischer Scientific, USA and used as received. Polyvinyl alcohol (PVA, ˜86% hydrolyzed and Mw=86,000 g/mol) and poly(methyl methacrylate) (PMMA, =15,000 g/mol) are purchased from Sigma Aldrich, USA.

### Methods

### CO₂-Assisted Impregnation of Silica Aerogel

Silica aerogels are prepared as described elsewhere using sol-gel and supercritical fluid processing¹⁹. These silica aerogels are highly porous carrier materials for the PFD. A high-pressure apparatus (Parr instruments, model 5500 with magnetic stirrer drive) is used for CO₂-assisted impregnation. Enough PFD is placed at the bottom of the vessel so that CO₂ remains saturated at the operating temperature and pressure¹⁸ of 25±1° C. and 900±20 psig during the impregnation period. Silica aerogel, dried at 100° C. for two hours prior to impregnation, is placed in a tea bag that is tied to the impeller shaft of the high-pressure apparatus so that the silica aerogel does not contact or soak in the liquid PFD. The PFD loading in the aerogels is determined gravimetrically.

### Coating PFD-Loaded Silica Aerogel Particles

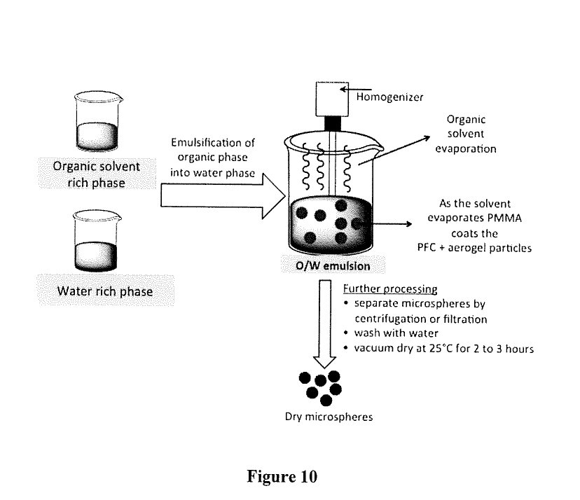

FIG. 10 shows a schematic diagram of the oil-in-water (O/W) emulsification and solvent evaporation method used to coat the PFD-loaded aerogel particles with PMMA. The particles, created by crushing the PFD-loaded aerogels into fine powder, are suspended in dichloromethane containing PMMA (1:1 wt/wt ratio of PMMA/aerogel) and PFD. The dichloromethane-rich suspension is emulsified into an aqueous solution (1:3 wt ratio) containing PVA that acts as a stabilizer. Dichloromethane is then evaporated from solution by continuously stirring at ambient conditions for four hours. The resultant microparticles are recovered from solution by first centrifuging the solution followed by rinsing the recovered particles twice with distilled water and then drying the particles under vacuum at 25° C. for three hours. In the remainder of this example, the PFD-loaded silica aerogels are referred to as uncoated aerogels and the PMMA-coated, PFD-loaded silica aerogels are referred to as coated aerogels.

### Aerogel Characterization

Nitrogen adsorption/desorption measurements are used to determine the aerogel surface area and pore size distribution. The pre-weighed aerogel sample is degassed in a vacuum at 120° C. for four hours prior to performing the nitrogen adsorption/desorption measurements at 77 K (Quantachrome Instruments, Nova® 2200e). The specific surface area of the aerogel is calculated with the multipoint BET model in the relative pressure range of 0.05 to 0.30. The pore size distribution is calculated with the BJH model using a desorption isotherm for a relative pressure of less than 0.35.

Thermal gravimetric analysis (TGA) (Perkin-Elmer USA, Model Pyris 1) is used to determine the PFD loading in the uncoated aerogels, where the sample is heated to 150° C. at a rate of 10° C./minute and held at this temperature for 60 minutes. TGA is also used to determine the PFD loading and the weight of PMMA for the coated aerogels by step-wise heating from 20 to 700° C. Inverted light microscopy and scanning electron microscopy (SEM) are used to determine the morphology of coated aerogels.

### Results and Discussion

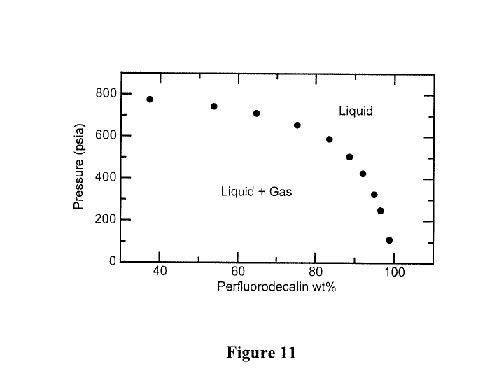

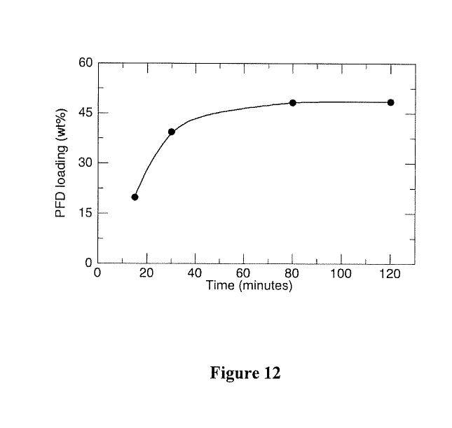

PFD is a hydrophobic and lipophobic compound, hence it is expected to be highly soluble in CO₂. FIG. 11 shows vapor-liquid equilibrium literature data for the PFD-CO₂ system¹⁸ 20° C., which suggests that operating the CO₂-assisted processing at 25° C. and 900±20 psia will be sufficient for an effective PFD impregnation process. FIG. 12 shows the effect of contact time on the amount of PFD loaded in the aerogels. As the contact time increases from 15 to 80 minutes, the PFD loading in the silica aerogels initially increases and ultimately reaches a plateau. Approximately 40-to-50 wt % of PFD is loaded into the aerogels at 25° C. and 900 psia.

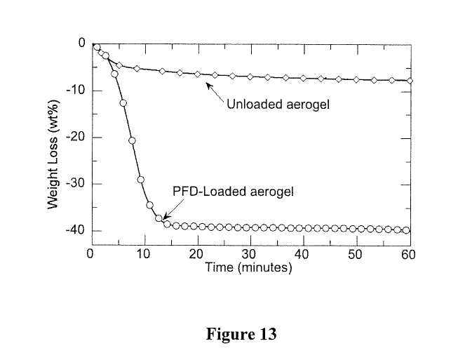

FIG. 13 shows the weight loss of unloaded aerogel and PFD-loaded, but uncoated aerogel determined by TGA. The unloaded aerogel loses ˜8 wt % which is water that evaporates at 150° C. The ˜40 wt % aerogel weight loss from the loaded, but uncoated aerogel exactly matches the amount of PFD in the aerogel as determined gravimetrically. The loaded PFD evaporates quickly from the aerogels when heated to 150° C. It should be noted that prior to loading with PFD, the aerogels are pre-heated at 100° C., so the weight loss obtained from the TGA is expected to be only from the PFD.

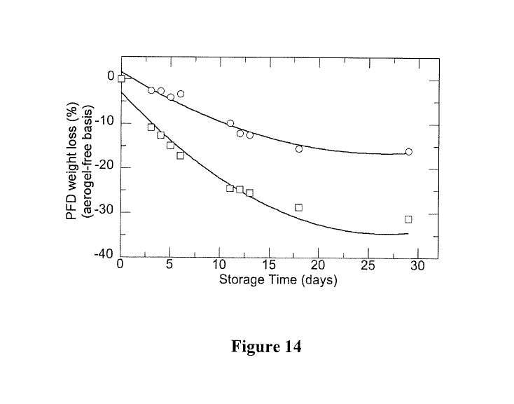

FIG. 14 shows the PFD shelf-life of the uncoated aerogels determined at 2-to-8° C. and 20-to-23° C. The uncoated aerogels lose ˜15% of the total loaded PFD (aerogel-free basis) when stored at 2-to-8° C. for 30 days, whereas aerogels stored at 20-to-23° C. lose twice as much total loaded PFD during the same time period. Given that the rate of PFD loss is fixed by the diffusion of PFD through the pores of the aerogel, a thin polymer coating on the loaded aerogel is expected to minimize the loss of PFD, and, hence, increase the shelf-life of the particles.

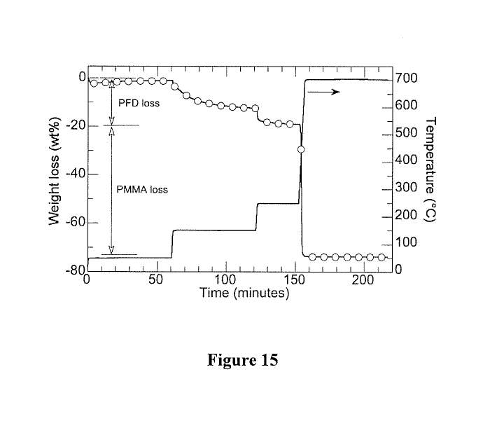

FIG. 15 shows the weight loss of coated aerogel when heated from 20 to 700° C. The coated aerogel contains ˜50 wt % PMMA, ˜20 wt % PFD (40 wt % on a PMMA-free basis), and ˜30 wt % silica aerogel based on the starting materials. The weight loss shown in FIG. 15 very closely matches the theoretical weight composition of the coated aerogel.

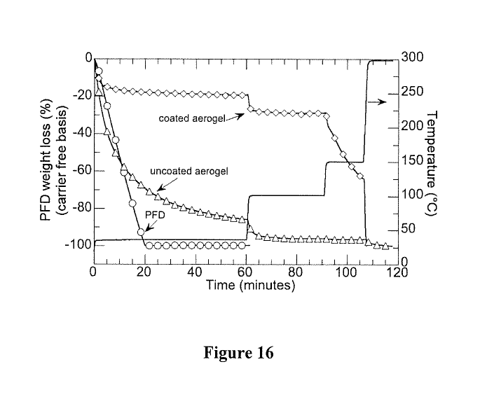

FIG. 16 compares the PFD weight loss (aerogel-free basis) from uncoated aerogel, coated aerogel, and pure PFD. All three sample are held at 37° C. for 60 minutes. During this period 20% of the available PFD is lost from the coated aerogel, more than 80% of the PFD is lost from the uncoated aerogel, and 100% of the pure PFD is lost. For the next 30 minutes the temperature is held at 100° C. During this next period another 10% PFD is lost from the coated aerogel and the remaining 20% PFD is lost from the uncoated aerogel. It is evident that the PMMA coating significantly reduces the evaporative loss of PFD.

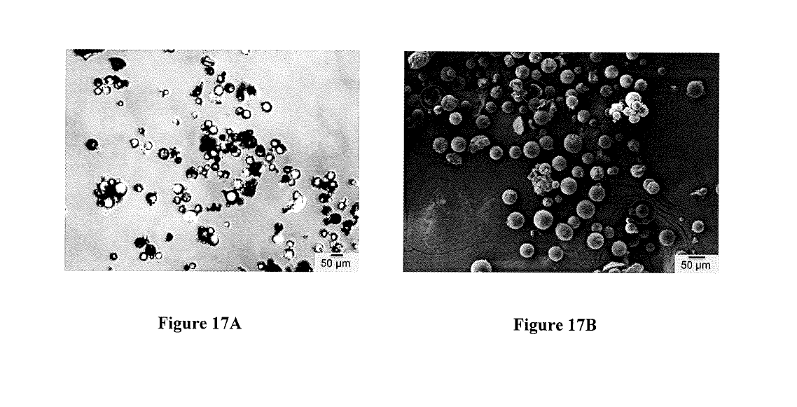

FIG. 17 shows the morphology of the coated aerogel. The inverted light microscopy image (FIG. 17A) shows that the bright PFD-loaded aerogel particles are encapsulated within a dark PMMA shell. FIG. 17B shows the SEM image that is used to further evaluate the morphology of the coated aerogels. These SEM results further confirm that PMMA coats the PFD-loaded aerogels and slows the evaporation of PFD from the coated aerogel as seen in FIG. 16. The red circles in the FIG. 17B highlight the broken PFD-filled PMMA microcapsules that are likely formed during the PMMA coating process.

### CONCLUSIONS

In this study, silica aerogels were successfully impregnated with PFD using subcritical CO₂ at mild operating temperatures and pressures. The PFD loading in the aerogels can be tailored between 18-to-50 wt % by manipulating the impregnation contact time. Maximum PFD loading in the aerogels is achieved at a contact time of 60-to-80 minutes. Shelf-life studies show that storing the PFD-loaded aerogels at 2-to-8° C. retains more than 85 wt % of the loaded PFD and thus storing in the refrigerator is preferred over room temperature. PMMA coating significantly improved the shelf-life of the PFD-loaded aerogels. The coated aerogels have potential applications in advanced wound care for sustained oxygen delivery.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

### REFERENCES

Throughout this application, various references describe the state of the art to which this invention pertains. The disclosures of these references are hereby incorporated by reference into the present disclosure.
1. C. Ji, A. Barrett, L. A. Poole-Warren, N. R. Foster, F. Dehghani, The development of a dense gas solvent exchange process for the impregnation of pharmaceuticals into porous chitosan, International journal of pharmaceutics, 391 (2010) 187-196.
2. S. G. Kazarian, G. G. Martirosyan, Spectroscopy of polymer/drug formulations processed with supercritical fluids: in situ ATR-IR and Raman study of impregnation of ibuprofen into PVP, International journal of pharmaceutics, 232 (2002) 81-90.
3. Z. Shen, G. S. Huvard, C. S. Warriner, M. Mc Hugh, J. L. Banyasz, M. K. Mishra, CO2-assisted fiber impregnation, Polymer, 49 (2008) 1579-1586.
4. B. S. K. Gorle, I. Smirnova, M. A. McHugh, Adsorption and thermal release of highly volatile compounds in silica aerogels, The Journal of Supercritical Fluids, 48 (2009) 85-92.
5. S. G. Kazarian, N. H. Brantley, B. L. West, M. F. Vincent, C. A. Eckert, In Situ Spectroscopy of Polymers Subjected to Supercritical CO2: Plasticization and Dye Impregnation, Appl. Spectrosc., 51 (1997) 491-494.
6. A. R. Berens, G. S. Huvard, R. W. Korsmeyer, F. W. Kunig, Application of compressed carbon dioxide in the incorporation of additives into polymers, Journal of Applied Polymer Science, 46 (1992) 231-242.
7. K. R. Ward, G. S. Huvard, M. McHugh, R. R. Mallepally, R. Imbruce, Chemical Oxygen Generation, Respiratory Care, 58 (2013) 184-195.
8. B. S. Harrison, D. Eberli, S. J. Lee, A. Atala, J. J. Yoo, Oxygen producing biomaterials for tissue regeneration, Biomaterials, 28 (2007) 4628-4634.
9. C. K. Sen, Wound healing essentials: Let there be oxygen, Wound Repair and Regeneration, 17 (2009) 1-18.
10. P. G. Rodriguez, F. N. Felix, D. T. Woodley, E. K. Shim, The role of oxygen in wound healing: A review of the literature, Dermatologic Surgery, 34 (2008) 1159-1169.
11. D. F. Roe, B. L. Gibbins, D. A. Ladizinsky, Topical dissolved oxygen penetrates skin: Model and method, Journal of Surgical Research, 159 (2010) e29-e36.
12. DAVIS, S. C., CAZZANIGA, A. L., RICOTTI, C., ZALESKY, P., HSU, L.-C., CREECH, J., EAGLSTEIN, W., MERTZ, P., Archives of Dermatology, Vol. 143, 2007, p. 1252
13. BAUER, J., ZAHRES, M., ZELLERMANN, A., KIRSCH, M., PETRAT, F., DE GROOT, H., MAYER, C., Journal of Microencapsulation, Vol. 27, 2010, p. 122
14. LOER, S. A., KINDGEN-MILLES, D., TARNOW, J., European Respiratory Journal, Vol. 20, 2002, p. 1499
15. PISANI, E., TSAPIS, N., PARIS, J., NICOLAS, V., CATTEL, L., FATTAL, E., Langmuir, Vol. 22, 2006, p. 4397
16. CHIN, L. S., LIM, M., HUNG, T. T., MARQUIS, C. P., AMAL, R., RSC Advances, Vol. 4, 2014, p. 13052
17. JANJIC, J. M., SHAO, P., ZHANG, S., YANG, X., PATEL, S. K., BAI, M., Biomaterials, Vol. 35, 2014, p. 4958
18. DIAS, A. M. A., CARRIER, H., DARIDON, J. L., PAMIES, J. C., VEGA, L. F., COUTINHO, J. A. P., MARRUCHO, I. M., Ind. Eng. Chem. Res., Vol. 45, 2006, p. 2341
19. SMIRNOVA, I., SUTTIRUENGWONG, S., ARLT, W., Journal of Non-Crystalline Solids, Vol. 350, 2004, p. 54

We claim:

1. A method for impregnation of at least one active ingredient into a carrier material, wherein said at least one active ingredient comprises an oxygen (O₂) carrier compound, said carrier material having a high free volume, empty or void space, or high porosity, comprising the steps of
placing said at least one active ingredient into a high-pressure apparatus;
pressurizing said high-pressure apparatus with a sub- or supercritical fluid at a pressure high enough to dissolve or disperse said at least one active ingredient in said sub- or supercritical fluid, said pressurizing step producing a fluid with at least one dissolved or dispersed active ingredient;
contacting the fluid with at least one dissolved active ingredient with said carrier material; and
recovering an impregnated carrier material.

2. The method of claim 1, wherein said O₂ carrier compound is selected from a peroxide compound or a perfluorinated compound (PFC).

3. The method of claim 2, wherein said PFC is perfluorodecalin (PFD).

4. The method of claim 1, further comprising the step of coating said impregnated carrier material with one or more of polymethyl methacrylate (PMMA) polyvinylacetate, PLGA copolymer, and PLA homopolymer.

5. The method of claim 1, wherein said carrier material is a synthetic polymer, inorganic or organic material, or inorganic or organic aerogel.

6. The method of claim 1, wherein said carrier material is selected from the group consisting of polyvinylpyrrolidone (PVP), polyethylene glycol (PEG), poly-2-ethyl-2-oxazoline, copolymers of ethylene and vinyl acetate, ethylene and methyl acrylate, lactic and glycolic acid, ter-block copolymers of ethylene oxide blocks followed by propylene oxide blocks followed by ethylene oxide blocks (PEO-PPO-PEO), granulated fumed silica, silica nanoparticles, cellulose acetate, silica aerogels, and polysaccharide-based aerogels.

7. The method of claim 6, wherein said carrier material is a polysaccharide-based aerogel, and said polysaccharide-based aerogel is selected from the group consisting of alginate, chitosan, pectin, and starch.

8. The method of claim 1, wherein said sub or supercritical fluid is selected from the group consisting of carbon dioxide (CO₂), ethane, propane, xenon, krypton, and fluorinated compounds that have a critical temperature ranging from −50° C. to 130° C.

9. The method of claim 1, further comprising maintaining said high pressure apparatus at a temperature of 25 to 40° C.

10. The method of claim 1, wherein said pressurizing step is maintained for 30 to 120 minutes.

11. A composition for the storage and controlled release of O₂ comprising a peroxide compound or PFC impregnated into a carrier material, wherein said carrier material is a synthetic polymer, inorganic or organic material, or inorganic or organic aerogel.

12. The composition of claim 11, further comprising one or more agents impregnated into said carrier material, said one or more agents being selected from the group consisting of antimicrobials, vasoactive peptides, anesthetic agents, hemostatic agents, anti-inflammatants, pain-reducing medication, additives to enhance blood clot strength, and growth factors.

13. A bandage for the treatment of wounds and burns comprising a peroxide compound or PFC impregnated into a carrier material, wherein said carrier material is a synthetic polymer, inorganic or organic material, or inorganic or organic aerogel, and wherein said carrier material is in contact on one side with an oxygen-impermeable membrane and on another side a first oxygen-permeable membrane.

14. The bandage of claim 13, further comprising a catalyst between said carrier material and said first oxygen-permeable membrane.

15. The bandage of claim 14, further comprising a second oxygen-permeable membrane or a removable oxygen-impermeable membrane between said carrier material and said catalyst.

16. The bandage of claim 15, wherein said first and second oxygen-permeable membrane is selected from the group consisting of medical grade films made of silicone, polyurethane, polyethylene and polyester.

17. The bandage of claim 16, wherein said medical grade film is made of silicone.

18. The bandage of claim 14, wherein said catalyst is selected from the group consisting of manganese dioxide, silver oxide, iron oxide, platinum oxide, copper oxide, potassium iodide, and catalase.

19. The bandage of claim 18, wherein said catalyst is manganese dioxide.

20. The bandage of claim 13, further comprising one or more agents impregnated into said carrier material, said one or more agents being selected from the group consisting of antimicrobials, vasoactive peptides, anesthetic agents, hemostatic agents, anti-inflammatants, pain-reducing medication, additives to enhance blood clot strength, and growth factors.

21. The bandage of claim 13, wherein said peroxide compound is selected from the group consisting of hydrogen peroxide, sodium peroxide, calcium peroxide, magnesium peroxide, urea hydrogen peroxide, sodium percarbonate, and poly(vinyl pyrrolidone)/hydrogen peroxide complex.

22. The bandage of claim 21, wherein said peroxide compound is hydrogen peroxide.

23. A method for treating wounds and burns comprising the step of administering to the wound or burn a composition comprising a peroxide compound or PFC impregnated into a carrier material, wherein said carrier material is a synthetic polymer, inorganic or organic material, or inorganic or organic aerogel, and wherein said peroxide compound or PFC delivers oxygen to the wound or burn.

24. The method of claim 23, further comprising one or more agents impregnated into said carrier material, said one or more agents being selected from the group consisting of antimicrobials, vasoactive peptides, anesthetic agents, hemostatic agents, anti-inflammatants, pain-reducing medication, additives to enhance blood clot strength, and growth factors.

25. The method of claim 23, wherein said composition is part of or associated with a bandage, wherein said carrier material is in contact on one side with an oxygen-impermeable membrane and on another side a first oxygen-permeable membrane, and said administering step includes applying said bandage to the wound or burn.

26. The method of claim 25, wherein said bandage is configured with a catalyst between said carrier material and said first oxygen-permeable membrane.

27. The method of claim 26, wherein said bandage further comprises a second oxygen-permeable membrane or a removable oxygen-impermeable membrane between said carrier material and said catalyst.

28. The method of claim 27, wherein said first and second oxygen-permeable membrane is selected from the group consisting of medical grade films made of silicone, polyurethane, polyethylene and polyester.

29. The method of claim 28, wherein said medical grade film is made of silicone.

30. The method of claim 26, wherein said catalyst is selected from the group consisting of manganese dioxide, silver oxide, iron oxide, platinum oxide, copper oxide, potassium iodide and catalase.

31. The method of claim 30, wherein said catalyst is manganese dioxide.

32. The method of claim 25, wherein said peroxide compound is selected from the group consisting of hydrogen peroxide, sodium peroxide, calcium peroxide, magnesium peroxide, urea hydrogen peroxide, sodium percarbonate, and poly(vinyl pyrrolidone)/hydrogen peroxide complex.

33. The method of claim 32, wherein said peroxide compound is hydrogen peroxide.

34. A bandage for the treatment of wounds and burns comprising microcapsules, creams, or gels loaded with a peroxide compound or PFC in contact on one side with an oxygen-impermeable membrane and on another side a first oxygen-permeable membrane.

35. The bandage of claim 34, further comprising a catalyst between said microcapsules, creams, or gels and said first oxygen-permeable membrane.

36. The bandage of claim 35, further comprising a second oxygen-permeable membrane or a removable oxygen-impermeable membrane between said microcapsules, creams, or gels and said catalyst.

37. The bandage of claim 36, wherein said first and second oxygen-permeable membrane is selected from the group consisting of medical grade films made of silicone, polyurethane, polyethylene and polyester.

38. The bandage of claim 37, wherein said medical grade film is made of silicone.

39. The bandage of claim 35, wherein said catalyst is selected from the group consisting of manganese dioxide, silver oxide, iron oxide platinum oxide, copper oxide, potassium iodide and catalase.

40. The bandage of claim 39, wherein said catalyst is manganese dioxide.

41. The bandage of claim 34, further comprising a carrier material positioned on either side of said microcapsules, creams, or gels.

42. The bandage of claim 34, further comprising one or more agents loaded into said microcapsules, creams, or gels, said one or more agents being selected from the group consisting of antimicrobials, vasoactive peptides, anesthetic agents, hemostatic agents, anti-inflammatants, pain-reducing medication, additives to enhance blood clot strength, and growth factors.

43. The bandage of claim 34, wherein said peroxide compound is selected from the group consisting of hydrogen peroxide, sodium peroxide, calcium peroxide, magnesium peroxide, urea hydrogen peroxide, sodium percarbonate, and poly(vinyl pyrrolidone)/hydrogen peroxide complex.

44. The bandage of claim 43, wherein said peroxide compound is hydrogen peroxide.

45. A method for treating wounds and burns comprising the step of administering to the wound or burn a first bandage comprising a catalyst and a second bandage comprising a peroxide compound impregnated into a carrier material, wherein said carrier material is a synthetic polymer, inorganic or organic material, or inorganic or organic aerogel, and wherein said carrier material is in contact on one side with an oxygen-impermeable membrane and on another side an oxygen-permeable membrane.
